(19) United States
(12) Patent Application Publication
(10) Pub. No.: US 2003/0021848 A1
(43) Pub. Date: Jan. 30, 2003

(54) Sustained percutaneous delivery of a biologically active substance

(76) Inventors: Matthew S. Johnson, Carmel, IN (US); Gordon McLennan, Zionsville, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/148,047

(22) PCT Filed: Nov. 29, 2000
(86) PCT No.: PCT/US00/32467

(51) Int. Cl. A61K 9/14; A61K 38/00

(52) U.S. Cl. 424/488; 514/2

(57) ABSTRACT

Disclosed are compositions and methods for sustained percutaneous delivery of a biologically active substance in a timed-release manner. For example, the compositions and methods can involve percutaneous injection into the tissue surrounding the adventitia of a treated artery at the time of angioplasty, thereby significantly lessening smooth muscle cell proliferation, and thus intimal hyperplasia (IH) and restenosis.

### CROSS-REFERENCE TO RELATED APPLICATIONS

The present application claims the benefit of U.S. application No. 60/167,834, filed on Nov. 29, 1999, which is hereby incorporated in its entirety by reference.

### TECHNICAL FIELD OF THE INVENTION

The present invention pertains generally to sustained release of a biologically active substance by percutaneous injection and immobilization within a polymer. For example, the biologically active substance can be delivered to the peri-adventitial surface of blood vessels (e.g., arteries).

### BACKGROUND OF THE INVENTION

Percutaneous balloon angioplasty has become an accepted treatment of stenotic (narrowed) arteries and veins. Unfortunately, this technique is limited by a high rate of failure. In this respect, angioplasty of a vessel (e.g., coronary or peripheral arteries) is frequently complicated by early recurrence of the stenosis, referred to as “restenosis,” thereby limiting the patency of the vessel. The rate of restenosis in vessels generally is inversely related to the size of the vessel, with higher rates, for example, in the femoral and infrapopliteal arteries than in the aorta and iliac arteries. By way of example, in the case of coronary arteries, approximately 30% to about 40% of stenoses treated with angioplasty recur within one year of the procedure.

Restenosis in any artery is thought to result mainly from a combination of vessel recoil, remodeling, and intimal hyperplasia. Recoil, which occurs over minutes, and remodeling, which occurs over days, typically are caused by contracting mechanical forces of the vessel wall. These forces may be overcome by placement of metal stents within the artery. However, the use of stents has not been fully satisfactory in inhibiting the onset of restenosis. While studies evaluating the effect of stents following coronary angioplasty found an approximately 30% reduction of angiographic restenosis in those patients who were stented, 20% of those patients who received stents still required revascularization. As such, it is believed that recoil and remodeling play a less significant role than intimal hyperplasia in restenosis. In the femoral and smaller peripheral arteries, stents have met with restenosis rates similar to those following angioplasty.

As a more significant factor in causing restenosis, intimal hyperplasia (IH) is a process in which the acute vascular injury produced by angioplasty incites a complex response, involving mitogen stimulation, platelet deposition, medial smooth muscle cell proliferation and migration to the intima, and extracellular matrix production. As a result of this response, the vessel lumen renarrows so as to cause restenosis. In several in vitro and animal models, certain agents have demonstrated some effect in reducing IH in small mammals, such as rats and rabbits. However, none of the studied agents or delivery methods has proven to be beneficial in clinical trials.

One such agent, heparin, has been shown (e.g., in vitro and in porcine and primate models) to have inhibitory effects on at least some components of intimal hyperplasia, including suppression of platelet degranulation, inhibition of smooth muscle cell (SMC) migration and proliferation, and modulation of the extracellular matrix surrounding vascular smooth muscle cells. Indeed, heparin has proven to be beneficial in decreasing restenosis in multiple animal models when given intravenously or subcutaneously. However, heparin delivered locally to the site of angioplasty has been shown to be more effective than intravenously administered heparin in decreasing SMC proliferation. Moreover, inasmuch as intimal hyperplasia is a dynamic process (e.g., smooth muscle proliferation and migration begin a few days following angioplasty, peaking at about a week, and continuing at an increased rate for about a month), effective control of this process requires that the heparin be administered continuously for several days. Studies comparing the effect of heparin, given at different intervals, and by different routes, upon the extent of IH following arterial injury in rats have found that, while intravenous administration of heparin yielded a decrease in IH, the most pronounced decrease in IH resulted from heparin administered via polymer matrices implanted adjacent to the adventitial surface of the injured arteries. These implanted matrices released heparin with near-zero order kinetics for several days. Also, local drug delivery has the potential advantage of producing a high concentration of the delivered drug at the desired site, in this case the vessel wall, while minimizing the amount of circulating drug and hence the side effects of that drug.

However, both intravenous and conventional local administration of heparin have proven to be clinically impractical. For example, continuous intravenous administration requires prolonged hospitalization, whereas matrix administration undesirably requires a surgical procedure as an adjunct to angioplasty. Hence, neither conventional method of heparin administration has met with success clinically.

Other approaches for decreasing IH include local brachytherapy, gene therapy, and catheter-based intravascular local delivery of bioactive agents. However, brachytherapy treatments have not enjoyed lasting results. Meanwhile, gene therapy still is in a formative stage and is yet to be fully developed inasmuch as it has proven difficult to identify optimal vectors while minimizing non-target negative effects. Furthermore, catheter-based intravascular local delivery techniques are limited by low delivery rates because of limitations of catheter design and because arterial flow can “wash away” the bioactive agent being delivered.

Accordingly, it will be appreciated from the foregoing that there exists a need for compositions and methods which permit sustained release of a biologically active substance by way of local delivery (e.g., percutaneously) to an internal locus. There is also a need for such compositions and methods that do not require a surgical procedure or an extended hospital stay associated with the delivery of the biologically active substance. It will also be appreciated that there exists a need for such compositions and methods which involve delivery in which the amount of drug circulating systemically is minimized, e.g., such that side effects relating thereto are minimized. It is an object of the present invention to provide compositions and methods satisfying at least one of these needs. These and other objects and advantages of the present invention, as well as additional inventive features, will be appreciated from the description of the invention provided herein.

### BRIEF SUMMARY OF THE INVENTION

The present invention provides compositions and methods that permit sustained release of a biologically active substance at a local site in an animal such as a mammal. In particular, a polymer having at least two charged portions of the same charge and a biologically active agent are delivered percutaneously (e.g., by way of a hypodermic needle) to a desired internal locus. Preferably, the polymer and the biologically active agent are combined while each is in liquid form (e.g., the polymer and biologically active agent themselves are liquids or are solids but are included in a liquid solvent, carrier excipient, vehicle, diluent, or the like). Multi-valent counter-ions (charged opposite to the charged portions of the polymer) also are included in the mixture in an amount sufficient to cross-link with the polymer, so as to form a matrix that contains the biologically active agent. The multi-valent counter-ions can be provided by the biologically active agent, or the counter-ions can be provided by an independent source. In some embodiments, the biologically active agent and the counter-ions are combined first, with the liquid polymer subsequently added to the mixture. Preferably, the matrix is allowed to set up, or harden, in vivo at a desired target location, although the matrix can be formed ex vivo if desired and then delivered (e.g., through an incision) to a desired internal locus. The biologically active agent then is released at the desired internal location in a sustained manner over a desired period of time, for example, by diffusion from the matrix.

Thus, in one aspect, the present invention provides a composition comprising a biologically active substance and a solid matrix capable of forming in vivo. The matrix comprises a biologically-compatible polymer having at least two charged portions of the same charge. The polymer is cross-linked with a multi-valent counter-ion. By way of example, and not by way of limitation, the ion equivalence (IE) ratio of the multi-valent counter-ion to polymer having at least two charged portions of the same charge can be in a range of from about 0.20 to about 2. The biologically active substance is contained within the solid matrix and can be released in a sustained manner over time (e.g., by diffusion).

In another aspect, the present invention provides a composition comprising a biologically-active substance and a matrix. The matrix comprises an alginate cross-linked with at least one biologically-compatible multi-valent cation. The biologically active substance is contained within the matrix from which it can be released in a sustained manner over a desired time period.

In yet another aspect of the present invention, a method of preparing a composition is provided. The method comprises providing a mixture comprising a biologically active substance and a biologically-compatible polymer having at least two charged portions of the same charge, such as, for example, an alginate anion or salt. The polymer is cross-linked with at least one biologically-compatible multi-valent counter-ion to produce a solid matrix that is capable of forming in vivo and that contains the biologically active substance. Preferably, the multi-valent counter ion is combined with the polymer in vivo, but, alternatively, the multi-valent counter-ion component can be combined with the polymer immediately prior to entry into the body (e.g., within one minute of entering the body, preferably, within a few seconds of entering the body). The multi-valent counter ion component can be combined with the biologically active substance prior to inclusion of the polymer, or the biologically active substance can be combined with the polymer prior to inclusion of the multi-valent counter-ion. For example, a biologically active substance/polymer mixture and the multi-valent counter-ion can be delivered via a hypodermic needle as described in U.S. Pat. No. 5,893,839, which is hereby incorporated in its entirety by reference. Other delivery approaches will be readily apparent to those of ordinary skill in the art.

The present invention will be more fully understood upon reading the following detailed description.

### DETAILED DESCRIPTION OF THE PREFERRED EMBODIMENTS

The present invention is predicated, at least in part, on providing compositions and methods suitable for permitting sustained release of a biologically active substance at an internal locus of a patient (e.g., an animal such as a mammal). In accordance with the present invention, a biologically active substance is contained within a matrix and released locally over a desired period of time. In preferred embodiments, the matrix is formed in vivo.

The matrix is prepared by cross-linking a biologically-compatible polymer. The polymer comprises at least two charged portions of the same charge (positive or negative). The polymer is cross-linked with a biologically-compatible multi-valent counter-ion, which has a charge opposite to the charge of the at least two charged portions of the polymer having the same charge. The multi-valent counter-ion is able to bond with two charged portions of the polymer, thereby creating the cross-linking effect. A sufficient quantity of counter-ions is thus provided to achieve the desired cross-linking with the polymer.

Prior to cross-linking, the polymer, biologically active substance, and multi-valent counter-ions, or their sources, all preferably are in liquid form. In this respect, each of these ingredients can be liquids themselves, and/or they can be carried in a liquid medium. By providing these ingredients in such a liquid form, they desirably can be delivered to an in vivo locus by percutaneous injection, e.g., via a needle. Once the polymer is combined with the multi-valent counter-ions, the setting up, or hardening, of the matrix will initiate. The formation of the matrix will occur as the flowability, or liquid properties, are lost, and will continue until movability is lost, as described in more detail in McLennan et al., “Kinetics of Release of Heparin from Alginate Hydrogel,” JVIR 2000, 11:1087-1094, which is hereby incorporated in its entirety by reference. The formation of the matrix in vivo, in accordance with preferred embodiments of the invention, refers to any point during the continuum when the flowability begins to be lost until the movability is lost. In this respect, even the flowability preferably is lost in vivo, but in some embodiments, the flowability can begin to be lost ex vivo, so long as the mixture is in a pourable state such that it can be percutaneously delivered (e.g., by injection via a needle), with completion of the matrix occurring in vivo. The composition typically can be in the form of a hydrogel, with water also present in the matrix.

The multi-valent counter-ions can be provided by the biologically-active agent or they can be provided by an independent source. For example, certain biologically active agents may include the requisite multi-valent counter-ions to achieve the cross-linking with the polymer. Strictly by way of example, proteins composed of positively charged amino acids (e.g., arginine and lysine), for example, alone or in combination with neutral amino acids, can provide the counter-ions to cross-link with the polymer having two or more negative charges. Preferably, the multi-valent counter-ions are provided by an independent source, for example, a salt.

To achieve matrix formation in vivo, pursuant to preferred embodiments of the invention, preferably the biologically-compatible polymer and the multi-valent counter-ions are combined in vivo, but in some embodiments, they are combined immediately (e.g., within a minute, preferably within 20 seconds more preferably within 10 seconds, even more preferably within a few seconds) prior to the percutaneous delivery to an internal locus. This is to avoid loss of flowability until the material is within the body. Of course, in cases where the biologically active substance provides the multi-valent counter-ions, the biologically active substance preferably is kept separate from the polymer until they enter the body or until immediately before percutaneous delivery. In embodiments where the multi-valent counter-ions are provided by an independent source, the ingredients can be combined in any suitable order so long as the multi-valent counter-ions and the polymer are not combined until the ingredients enter the body or until immediately prior to percutaneous delivery. For example, the biologically active substance and the multi-valent cations, or their sources, can be combined first with the polymer subsequently added, or, alternatively, the biologically active substance and the polymer can be combined first, with the multi-valent cations added subsequently. The amount of time that elapses after combining the biologically active substance and the multi-valent cations but prior to adding the polymer, or the amount of time that elapses after combining the polymer and the biologically active substance but prior to adding the multi-valent cations, is not critical.

In some embodiments, it may be desired to form the matrix ex vivo. Strictly by way of example, in operations for performing dialysis or arterial bypass grafts, a biologically active agent, such as heparin, can be immobilized within a matrix according to the invention, and provided along an anastomosis so as to permit sustained release of the biologically active agent over time, for example, to avoid narrowing of the out-flow vein associated with the dialysis graft or the artery at the arterial bypass graft anastomosis.

While not wishing to be bound by any particular theory, after the biologically agent is immobilized within the solid gel matrix, the biologically active agent will be released in a sustained manner by way of diffusion through pores in the matrix. It is also believed that the release of the agent can typically occur in three phases, as discussed in McLennan et al., supra. Because the matrix is a hydrogel, water also will leave the matrix such that the matrix preferably shrinks in size and preferably degrades over time, following release of the drug. The matrix is selected so that it is biologically-compatible such that, even if it does not degrade (e.g., because it absorbs ambient water in the body), it is not harmful to the body.

The present invention can encompass any of a variety of biologically active agents, such as, for example, a drug, a gene, a nucleic acid, a protein, an antibody, a fatty acid, a carbohydrate, a vector, a cell, or the like, and combinations thereof. Any suitable biologically active substance can be utilized so long as it can be contained within the matrix in efficacious amounts, as will be appreciated by one of ordinary skill in the art, and so long as it can be released in a suitable time frame, as also will be appreciated by one of ordinary skill in the art. Exemplary drugs include, for example, heparin or derivatives thereof (including low molecular weight forms of heparin such as nadroparin, enoxiparin or derivatives thereof), anti-cancer agents, such as, for example, paclitaxel, adriamycin, cisplatin, or the like. Carbohydrates such as starch, saccharides (mono-, di-, or poly-), or the like also can be selected as the biologically active substance. For example, suitable carbohydrates include, but are not limited to, sucrose, glucose, lactose, maltose, fructose, cellobiose, glycosaminoglycan, or the like. Exemplary proteins include, but are not limited to, angiogenic proteins such as vascular endothelial growth factor (VEGF), chemotactic proteins such as monocyte chemotactic proteins (e.g., MCPI), and the like. Exemplary antibodies include, for example, anti-vascular endothelial growth factor (anti-VEGF), and the like. Suitable fatty acids include, but are not limited to, triglycerides, lipoproteins such as HDL, or the like. The biologically active agent also can be in the form of a vector (e.g., as a means to deliver a gene to a cell), such as, for example, an adenovirus, plasmid, retrovirus, or the like. Exemplary cells that can be utilized as the biologically-active agent include, but are not limited to, natural killer cells (NK cells), T cells, B cells, red blood cells, macrophages, white blood cells, and the like. It will be appreciated that one or more of each type of biologically active substance, or combinations of different types of biologically active agents, can be utilized in the practice of the invention.

The polymer can be any suitable biologically-compatible polymer that has multiple charged portions of the same charge and which can be cross-linked with a multi-valent counter-ion. It is noteworthy that the multiple charged portions of the same charge can be anionic or cationic, with the multi-valent counter-ions then being the opposite charge. For example, if the multiple charged portions of the same charge of the polymer are anionic, then the multi-valent counter-ion would be cationic and if the multiple charged portions of the polymer are cationic, then the multi-valent counter-ion would be anionic. In preferred embodiments, the multiple charged portions of the polymer are anionic such that the multi-valent counter-ions are cationic.

The multi-valent counter-ions can be of any suitable type so long as they are biologically-compatible and capable of cross-linking with the polymer having the multiple charged portions of the same charge. While not wishing to be bound by any particular theory, it is believed that a single multi-valent ion can bond to two separate oppositely-charged groups on a polymer to create cross-linking. The multi-valent counter ions can be positively charged or negatively charged. To correspond to the preferred negatively charged multiple charged portions of the polymer, the multi-valent counter-ions preferably are cations. By way of example, and not by way of limitation, suitable multi-valent cations include, but are not limited to, calcium, magnesium, manganese, or the like, or combinations thereof.

An exemplary polymer useful in the practice of the invention is an alginate. Alginates are a family of linear polymers of linked β-D-mannuronic acid and α-L-guluronic acid. Alginates, as described herein, refer to derivatives of alginic acid, or the anionic portion thereof. Exemplary alginates include, for example, potassium alginate, sodium alginate, propylene glycol alginate, and the like. A preferred alginate is sodium alginate, which includes the following monomeric unit:

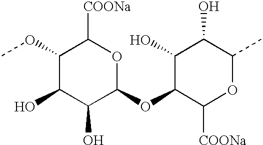

As will be appreciated by one of ordinary skill in the art, sodium alginate is water soluble. Because sodium is monovalent, it bonds to only one COO⁻ unit on the alginate at a time. By reacting the sodium alginate with a salt containing a multi-valent cation, the multi-valent cation will cross-link with the alginate to form the desired matrix. In this respect, it is believed that when multi-valent (e.g., divalent) ions, such as, for example, calcium, are added, cross-linking occurs at the carboxyl side chains thereby forming an insoluble three dimensional matrix. When the biologically active substance (e.g. heparin) is mixed with the alginate prior to cross-linking, the biologically active substance becomes immobilized within the solid matrix of the alginate hydrogel. The release of the biologically active substance from the gel is slowed because the drug is incorporated within the gel and must be released from it.

Thus, in accordance with an aspect of the invention, a polymer having the desired characteristics described herein, such as an alginate, is dissolved in water or other suitable biologically-compatible solvent. For example, sodium alginate can be selected. The sodium alginate can be present in any suitable amount, such as, for example, a concentration of from about 0.5% to about 4% by weight of the solution, preferably from about 1% to about 3% by weight, and more preferably about 2% by weight. The liquid solution of sodium alginate then can be combined with a biologically active substance, which preferably is either a solid or liquid that is carried in a liquid medium. The biologically active agent can be provided as a liquid or solvent in a liquid medium, in any suitable amount. As will be appreciated by one of ordinary skill in the art, the amount of biologically agent will depend on the nature of the agent and the type of treatment involved. Strictly by way of example, where the biologically active agent is a clot inhibitor such as heparin, the heparin can be in a range of, for example, from about 1.000 to about 10,000 units of activity, preferably from about 2,000 to about 6.000 units, more preferably, about 4,000 units. The mixture of the biologically active substance and the sodium alginate is combined with a salt containing multi-valent cations, such as, for example, calcium, magnesium, manganese, or the like. A preferred salt is calcium gluconate. Alternatively, if desired, the calcium gluconate can be combined with the biologically active substance prior to including the sodium alginate in the mixture. In either event, the salt containing the multi-valent cations is provided in a liquid medium in any suitable amount, e.g., in an amount of from about 0.2 M to about 0.8 M, preferably from about 0.4 M to about 0.6 M, more preferably, about 0.46 M.

Notably, in accordance with preferred embodiments of the invention, the ion equivalence (IE) ratio of the multi-valent counter-ion to the polymer having at least two charged portions of the same charge is from about 0.2 to about 2. Accordingly, the amounts of multi-valent ion and polymer that are combined preferably result in a matrix exhibiting an IE ratio of from about 0.2 to about 2, as will be appreciated by one of ordinary skill in the art. Ion equivalence ratio is described, for example, in McLennan et al., supra. Preferred compositions according to the invention having a multi-valent counter-ion:polymer IE ratio of from about 0.2 to about 2 exhibit surprising and unexpected advantages in optimizing the matrix formation characteristics, the amount of biologically active substance contained within the matrix, and/or the release rate kinetics of the biologically active substance.

Within the preferred IE ratio range of 0.2 to about 2, the amount of the multi-valent counter-ion used to cross-link the polymer can be varied in order to adjust the gel formation characteristics, the amount of biologically active substance immobilized within the gel, and the release rate of the biologically active substance from the gel (e.g., preferably, zero order kinetics), as desired. As will be appreciated by one of ordinary skill in the art, the size (e.g., molecular weight) of the biologically active agent, the charge distribution of the biologically active agent, and the desired time distribution of release of the biologically active agent all can impact the desired multi-valent ion: polymer IE ratio. For example, smaller biologically active agents (e.g., having a molecular weight less than about 1 kD) tend to diffuse out of the polymer matrix at faster rates such that a higher multi-valent counter-ion: polymer ratio within the preferred range of 0.2-2 may be desired to slow down the rate of diffusion of the biologically active agent from the polymer matrix. Conversely, larger biologically active agents (e.g., having a molecular weight of at least about 50 kD) may diffuse from the polymer matrix slowly such that a lower multi-valent ion: polymer ratio within the preferred range of 0.2-2 may be desired to enhance the rate of diffusion of the biologically active agent from the polymer matrix.

By way of example, in some embodiments, the multi-valent counter-ion: polymer IE ratio is from about 0.25 to about 2, sometimes from about 0.25 to about 1.2, sometimes from about 0.25 to about 0.8, sometimes from about 0.4 to about 0.7, sometimes from about 0.5 to about 0.6. Strictly by way of example, in the case of heparin, the multi-valent ion preferably is calcium and the polymer is an alginate, in which the IE ratio falls preferably within these ranges, with an optimum calcium:alginate IE ratio of about 0.58.

The composition according to the invention can be delivered for any of a variety of prophylactic and/or therapeutic treatments. In addition, the present invention can be utilized to deliver biologically active agents to any of a variety of loci within the body. In these respects, the invention can be practiced at any location and for any in vivo purpose so long as the matrix forms and contains the biologically active agent and then permits its sustained release. It will be appreciated that the total amount of matrix that is delivered will vary, depending on the size of the locus in which treatment occurs, as well as the type of biologically active agent and the type of treatment. For example, to treat a mass (e.g., cystic or a tumor), the size of the mass can be determined and then the appropriate quantity of hydrogel according to the invention can be permitted to set up (following percutaneous delivery of the ingredients or the hydrogel itself) in and/or around the mass. Strictly by way of example, in the context of heparin contained within a matrix of alginate cross-linked with calcium used to treat the peri-adventitial area of a blood vessel, the amount of the composition delivered will vary depending upon the size of the vessel, but can be, for example, from about 0.5 ml to about 4 ml, preferably from about 1 ml to about 3 ml. more preferably about 2 ml.

An exemplary locus within the body in which the composition of the invention can be delivered and/or allowed to set up is the adventitial surface or periadventitial area of a blood vessel (e.g., an artery), for example, to prevent or treat restenosis. Delivery of the composition of the invention to the adventitial or periadventitial area of the vessel is advantageous because the delivered agent is not exposed to blood flow which otherwise might cause the composition to flow away from the desired locus. In this respect, the composition according to the invention can be delivered all around a vessel and may remain in contact with the vessel for an extended period. Additionally, recent studies suggest that the adventitia may play a major role in the development of intimal hyperplasia. Thus, adventitial drug delivery according to embodiments of the invention is advantageous as compared with intravascular delivery.

In accordance with an aspect of the present invention, by using flouroscopic guidance, a polymer (e.g., alginate) mixed with a biologically active substance (e.g., heparin) can be injected into a mammal immediately adjacent to a vessel (e.g., the carotid artery, femoral artery or the like). For example, unfractionated heparin is a mixture of glycosaminoglycans derived from swine or bovine intestinal mucosa. Injection of a multi-valent counter-ions, such as calcium, or a source thereof, immediately thereafter causes cross-linked polymerization of the polymer into a solid hydrogel that immobilizes the biologically active substance and causes sustained release.

The following examples further illustrate the present invention, but should not be construed as in any way limiting its scope.

### EXAMPLE I

This example demonstrates that heparin delivery can be sustained for 21 days using peri-adventitially injected heparin immobilized in a cross-linked alginate hydrogel.

Particularly, one femoral and both carotid arteries of 11 outbred swine were angioplastied to 20% over dilation. 0.2 ml of heparin, suspended in 1.6 ml of a 1% sodium alginate solution, was injected through a 20 gauge needle into the peri-adventitial space at the site of angioplasty. 0.2 ml of calcium gluconate was then injected to cross-link the alginate. In each animal, one injection was of tritated heparin (2 μCi/4000U), one injection was of 4000 U unlabeled heparin, and one injection was of 2000 U flouro isothiocyanate (FITC)-labeled heparin. Two animals were sacrificed acutely; 1 animal was sacrificed at 1 day; and 2 animals were sacrificed at 3, 7, 14, and 21 days. Vessels that received tritiated heparin were dissected, homogenized and scintillated. Vessels that received unlabeled heparin were dissected and homogenized, and heparin was extracted from them for analysis with high performance liquid chromatography (HPLC). Vessels that received FITC-labeled heparin were analyzed histologically and with fluorescent microscopy. The right and left iliac arteries were used as control vessels for scintillation, HPLC, and histology.

HPLC was not sensitive enough to give reliable quantification of the amount of heparin extracted from blood vessels. While liquid scintillation demonstrated variability in the amounts of heparin recovered, this assay demonstrated that heparin was delivered to the arterial wall at each time point. The average amount of heparin recovered at all time points was 12 times that of the recovery from control vessels. At 21 days, 0.5 and 0.1 units of heparin were present within the artery at the angioplasty site.

Accordingly, it was shown that heparin, percutaneously delivered within a cross-linked alginate hydrogel, was continuously released to the arterial wall for at least 21 days following its administration.

### EXAMPLE II

This example will demonstrate percutaneous administration of heparin-laden alginate to angioplastied carotid arteries of swine.

Percutaneous administration of heparin-laden alginate to angioplastied carotid arteries was performed in 10 juvenile outbred swine (30-40 kg) in the following manner:

Pre-induction anesthesia was obtained with an IM injection of Ketamine (28 mg/kg) and Atropine (0.04 mg/kg). Following intubation, each pig was placed supine on the fluoroscopy table. General anesthesia was maintained with Isoflurane. Via groin cutdown, an 8F vascular sheath was introduced into a common femoral artery. Heparin, 100U/kg, was administered IV. Bilateral carotid digital subtraction arteriography (DSA) and intravascular ultrasound (IVUS) was performed, and the latter used to measure carotid arterial cross-sectional area and diameter. One carotid artery was dilated to 120% of its measured diameter with an appropriate angioplasty balloon. Using fluoroscopic guidance, a needle (18-21 g) was introduced through the skin of the mid-neck to just outside of the angioplastied segment of carotid artery. 1.8 ml of heparin-laden alginate (0.2 ml heparin [4000U] suspended in 1.6 ml of 1% sodium alginate, yielding an 0.58 IE ratio) was injected into the tissue surrounding the artery (“peri-adventitial tissue”). Using the same needle, 0.2 ml calcium gluconate was injected at the same site. The site of administration was marked on the pig's skin and noted on DSA images obtained following administration. Following 120% overdilation angioplasty of the contralateral carotid artery at the same level, unladen alginate was injected into the tissues surrounding that artery and cross-linked with calcium gluconate, allowing that artery to serve as a control. Following arteriography of that carotid artery and marking of the site of injection, the femoral sheath was removed, the femoral artery ligated, and the groin incision closed in layers. The pig was recovered from anesthesia and then transferred to the animal care facility.

Each pig was returned to the angiography suite 28 days following the angioplasty/gel injection procedure. Arteriography and IVUS was repeated, with measurement of cross sectional diameters (DSA, IVUS) and cross-sectional areas (IVUS) of the carotid arteries at, proximal to, and distal to the angioplasty sites bilaterally. Bony landmarks and a hemostat clamped to the pig's skin prior to the DSA study was used to assist in localization of each angioplasty site during explantation. Additionally, a Cope suture-anchor (Cook, Inc., Bloomington, Ind.) was inserted percutaneously adjacent to the artery at the angioplasty site to facilitate localization of the vessel to be harvested. The carotid arteries was isolated with 3-0 silk ties well above and below the site of angioplasty and the isolated segment was perfusion fixed with 10% formalin. The carotid arteries and surrounding tissues was explanted en bloc and visually inspected for evidence of inflammation and calcium deposition. The angioplasty sites was marked with suture placed 10 mm above their cephalad extent prior to placement of the specimens into formalin. The relationship of that segment of carotid artery that had the area of maximum stenosis demonstrated by IVUS to the suture was noted. For subsequent BrdU and KI-67 staining, an iliac artery was explanted to be used as a negative control, and a portion of intestine was explanted to be used as a positive control.

Histologic evaluation, including BrdU and KI-67 will be performed. PCNA and KI-67 allow differentiation of proliferating from quiescent smooth muscle cells. PCNA stains proliferating nuclei, whereas KI-67 stains endogenous proteins in all cells not in the GO phase of the cell cycle. As such, the two studies are complementary. The examining pathologist will be blinded as to the treatment versus the control side. The percentages of proliferating smooth muscle cells will be compared from treatment to control. Measurement of luminal narrowing and of the ratio of intimal to medial volume will be performed, and compared to measurement of luminal narrowing as demonstrated by DSA and IVUS.

With respect to statistics, the treatment side will be determined by sealed envelope. The difference between the heparin-treated and placebo side values will be calculated for the proportion of smooth muscle cells that are stained and all inference will be done on the differences to account for any possible pig-to-pig variation. Using 10 pigs, a 95% confidence interval for the difference will be 1.24 times the standard deviation of the difference. If the differences are not normally distributed, then a transformation (square root or logarithm) will be used to try to make the variable(s) normally distributed before the calculation of the confidence intervals. If this does not result in normally distributed data, then we will use the non-parametric Wilcoxon signed rank test. Previous data have shown that the standard deviation for the difference in proportions of proliferating cells in unpaired data is approximately 20%. If we are interested in detecting a difference at least as large as 20%, then 10 pigs will give us 80.3% power to detect a difference when using a paired t-test at the 5% level (two-sided). If there is positive correlation between the two sides of a given pig (and we expect it to be somewhat positive), then the standard deviation of the paired differences will be smaller, resulting in a higher power.

### EXAMPLE III

This example demonstrates optimum release rates of unfractionated heparin from sodium alginate hydrogels crosslinked with varying amounts of calcium gluconate.

Particularly, six hydrogels, each composed of 0.16 mEq sodium alginate and 4000 units unfractionated heparin, were crosslinked (polymerized) with varying amounts of calcium gluconate to yield ion equivalence (IE) ratios (calcium:alginate) of 0.2, 0.4, 0.58, 0.8, 1.0, or 1.2. Two ml of normal saline were placed on top of each gel and allowed to remain in contact for up to 10 days. At set time intervals, the eluents were removed, replaced with the same quantity of normal saline, and filtered. Each filtered eluent was sampled with size exclusion high performance liquid chromatography (HPLC).

The gels created with 0.2 and 0.4 equivalence ratios were partially liquid at 24 hours; the other gels became solid within 10 minutes. The gel with IE ratio of 0.58 took the longest time to become solid but it immobilized the most heparin initially and it had the slowest release rate over the 10 day observation period. At 10 days, between 5.5% and 9.8% of the heparin initially immobilized was retained in the gel.

Accordingly, this hydrogel shows promise as a vehicle for in vivo perivascular heparin delivery. The 0.58:1 IE ratio hydrogel had the slowest release rate and the greatest immobilization despite its longer cross-linking time. The higher initial release of heparin from the higher IE ratio gels suggests that the greater crosslinking in those gels excludes more heparin from incorporation in the gel.

### EXAMPLE IV

This example compares the in vitro gel formation characteristics, the immobilization of heparin, and the release rates of heparin from gels created with varying amounts of cross-linking with calcium ions. This allowed us to choose an optimum concentration of calcium to use for our in vivo experiments. In the process of analyzing these gels, it was necessary to develop an assay for heparin in the presence of alginate.

Specifically, heparin (20,000 units/ml, Fujisawa USA, Deerfield, Ill.) was diluted in 0.9% sodium chloride USP to concentrations of 5, 10, 25, 50, 80, 100, 250, 500, 800, and 1000 units/ml. 100 μl samples of heparin at each concentration were analyzed with a Varian 9012 High Performance Liquid Chromatography (HPLC) pump (Varian Chromatography Systems, Walnut Creek, Calif.) with a Shodex OHpak SB-804 HQ column (Phenomenex, Inc, Torrence, Calif.), without a guard column, at a flow rate of 1.0 ml/min, pressure of 26 atm, with a 0.5M NaCl solution buffered to pH=8.9 with sodium tetraboratedecahydrate (0.01 M). The total run time was 40 minutes. Absorbances were measured using a Varian 9050 UV detector (Varian Chromatography Systems, Walnut Creek, Calif.) set at 206 nm. Absorbance height and area of the heparin peak were recorded and a calibration curves of peak height versus heparin concentration and area versus heparin concentration were generated.

Hydrogels were created in glass vials (23 mm diameter, 85 mm high, 35.3 ml) using 1.6 ml (0.16 mEq) of 2% aqueous solution of sodium alginate (Pronova LVG ultrapure, Pronova biopolymer, Gaustadalleen 21, N-0371, Oslo, Norway) (0.1 mEq/ml), 0.2 ml of Heparin sodium (4000 units) and 0.2 ml (0.093 mEq), 0.275 ml (0.128 mEq), 0.344 ml (0.16 mEq), 0.413 ml (0.192 mEq) of calcium gluconate (American Reagent Laboratories, Inc., Shirley, N.Y.)(0.465 mEq/ml). Three hydrogels were created with each concentration of calcium gluconate for a total of 12 hydrogels, as seen in Table 1, wherein 3 of each Gel were made for a total of 12 Gels.

TABLE 1

| Gel: | GH-03 (1, 2, 3) | GH-04 (1, 2, 3) | GH-05 (1, 2, 3) | GH-06 (1, 2, 3) |
|---|---|---|---|---|
| Alginate | 0.16 mEq | 0.16 mEq | 0.16 mEq | 0.16 mEq |
| (0.1 mEq/ml) | | | | |
| Heparin | 2000 Units | 2000 Units | 2000 Units | 2000 Units |
| (20,000 U/ml) | | | | |
| Calcium | 0.093 mEq | 0.128 mEq | 0.16 mEq | 0.192 mEq |
| (0.465 mEq/ml) | | | | |
| Ca:Alg ratio | 0.58 | 0.8 | 1.0 | 1.2 |

The time to loss of flowability (ability to move and change shape as a liquid) and the time to loss of mobility (ability to move at all) were recorded for all gels. Two additional hydrogels were created with 0.14 ml (0.064 mEq) and 0.07 ml (0.032 mEq) of calcium gluconate but these two gels were only partially gelled at 24 hours.

After gelling overnight, 2 ml of 0.9% sodium chloride were placed carefully on the top of the formed gel. The eluent was removed, filtered, and 100 microliters were injected into the HPLC for analysis. The remaining eluent was stored in a vial. 2 ml of fresh 0.9% sodium chloride were then replaced on the surface of the gel and allowed to remain in contact with the hydrogel for 1 hour prior to analysis with HPLC. This process was repeated at 2, 5, 8 hours and every 24 hours for 10 days for a total of 14 samples per hydrogel. HPLC was performed on 100 microliter samples of the eluent from each gel at each time point as described above.

The amount of heparin in the eluent at each time point was calculated based on the calibration curve of peak height versus heparin concentration. The amount of heparin in the eluent at time 0 was considered the amount of heparin excluded from the gel. This was recorded and subtracted from the amount of heparin used to create the gel to determine the amount of heparin immobilized in the gel. The amount of heparin in the eluent at each subsequent time period was recorded and divided by the amount of heparin in the gel to determine the percentage of release from the gel at each time point. Linear regression was performed on the percent release data from 0 to 8 hours. 8 to 96 hours, and 4 to 10 days. The slopes of these regressions were compared to assess the differences in the rate of heparin release during these three time periods. The percent released at each time point was then divided by the contact time to yield an hourly release rate for each gel at each time point and plotted versus time for each gel. The data were analyzed with multivariate analysis using a generalized linear model with the inverse link function because the inverse of heparin concentration had a linear relationship with contact time. In a secondary analysis, the rate of heparin release for sets of periods of varying lengths (2 periods of 1 hour, 2 periods of 3 hours, 1 period of 16 hours and 9 periods of 24 hours) was examined using analysis of variance.

Calibration curves of peak height versus concentration and area under curve versus concentration were generated, with the peak height used to determine heparin concentrations in test samples due to its slightly higher linear correlation.

Gel formation characteristics varied by the ratio of calcium to alginate. The two gels created with calcium to alginate ratio of 0.2 and 0.4 remained liquid at 24 hours. Heparin release from these gels was not studied. The gel with the calcium:alginate ratio of 0.58 lost its flowability 10 minutes after the addition of calcium. It took 55 minutes for the gel to lose its mobility. Gels with higher amounts of calcium lost their flowability and became solid faster but they also immobilized less heparin. These data are summarized in Table 2.

TABLE 2

| Equivalence ratio | Time to loss | Time to loss | Per Cent Heparin |
|---|---|---|---|
| Calcium:Alginate | of flowability | of mobility | excluded from Gel |
| 0.2 | >10 days | >10 days | |
| 0.4 | 10 days | >10 days | |
| 0.58 | 10 minutes | 55 minutes | 2.1% |
| 0.8 | 5 minutes | 55 minutes | 4.5% |
| 1.0 | 2 minutes | 39 minutes | 9.2% |
| 1.2 | 1 minute | 35 minutes | 15.6% |

The percent heparin released and the hourly released rates are illustrated in Tables 3a & 3b.

TABLE 3a
Percent Release of Heparin from Calcium Alginate Gels

Calcium:Alginate Ratio

| Time | 0.58 | 0.8 | 1 | 1.2 |
|---|---|---|---|---|
| 0 | 2.1 | 4.5 | 9.2 | 15.6 |
| 1 | 6.7 | 8.05 | 8.35 | 12.3 |
| 2 | 10.9 | 13.8 | 13.8 | 19.2 |
| 5 | 17.8 | 21.45 | 22 | 27.2 |
| 8 | 23.6 | 27.35 | 28.3 | 33.8 |
| 24 | 35.7 | 38.35 | 41.4 | 46 |
| 48 | 49.6 | 53.35 | 55.3 | 59.9 |
| 72 | 62.5 | 63.75 | 65.5 | 70 |
| 96 | 69.7 | 72.15 | 72.9 | 77.1 |
| 120 | 76.7 | 77.45 | 77.7 | 81.4 |
| 144 | 81 | 82.35 | 81.5 | 85.3 |
| 168 | 85.4 | 85.6 | 84.4 | 88.1 |
| 192 | 88 | 88.25 | 86.7 | 90.3 |
| 216 | 90.1 | 90.55 | 88.8 | 92.3 |
| 240 | 92.2 | 92.3 | 90.4 | 94.5 |

TABLE 3b
Hourly Heparin Release (% release/hr)

Calcium:Alginate Ratio

| Time | 0.58 | 0.80 | 1.00 | 1.20 |
|---|---|---|---|---|
| 1 | 6.70 | 8.05 | 8.35 | 12.30 |
| 2 | 4.15 | 5.75 | 5.40 | 6.85 |
| 5 | 2.30 | 2.55 | 2.73 | 2.68 |
| 8 | 1.93 | 1.97 | 2.10 | 2.18 |
| 24 | 0.76 | 0.69 | 0.82 | 0.76 |
| 48 | 0.58 | 0.63 | 0.58 | 0.58 |
| 72 | 0.54 | 0.43 | 0.43 | 0.42 |
| 96 | 0.30 | 0.35 | 0.31 | 0.30 |
| 120 | 0.29 | 0.22 | 0.20 | 0.18 |
| 144 | 0.18 | 0.20 | 0.16 | 0.16 |
| 168 | 0.18 | 0.14 | 0.12 | 0.12 |
| 192 | 0.11 | 0.11 | 0.09 | 0.09 |
| 216 | 0.09 | 0.10 | 0.09 | 0.09 |
| 240 | 0.09 | 0.07 | 0.07 | 0.09 |

The release characteristics of heparin varied by calcium:alginate ratio with the release rate being highest for the 1.20 ratio and lowest for 0.58 (P=0.03). There was some evidence that rate of change of the release rate differed between gels (P=0.075). Table 4 is a summary of 2 statistical models generated by multivariated regression.

TABLE 4
Estimated reciprocal of release rates as a function of time
(measured in hours)

| | |
|---|---|
| 1/Release | Model |

| Ca/alginate ratio | Main Effect | Interaction Model |
|---|---|---|
| 0.58 | 0.134 + 0.0411 time | 0.149 + 0.0367 time |
| 0.80 | 0.098 + 0.0411 time | 0.101 + 0.0399 time |
| 1.0 | 0.096 + 0.0411 time | 0.080 + 0.0435 time |
| 1.2 | 0.056 + 0.0411 time | 0.048 + 0.0448 time |

The main effect model assumes that the slope is the same for all gels and differences in release rate are reflected only in the intercept. Because the model is based on the reciprocal of the release rate, the larger intercept for the 0.58 gel indicates that this gel has the slowest release rate over the 10 day observation period. The interaction model allows the slopes to be different for each gel. In this model, the intercept is higher for the 0.58 gel but the slope is lower. This indicates that the rate of heparin release is initially slower for the 0.58 gel but the release rate decreases more slowly than the other gels over the 10 day observation period. In a secondary analysis, we have found that the rates of heparin release were also different when we examined time periods of equal lengths. In 1-hour and 3-hour long intervals, release rates were different among the gels with varied calcium:alginate ratio (i.e., P=0.0013 and P=0.014, respectively). In 2 periods of 1-hour length, release rate was fastest for the gel with calcium:alginate ratio of 1.20, and no different for the other concentrations. In 2 periods of 3-hour length, release rates for ion equivalent ratios of 1.00 and 1.20 were larger than the release rate for ion equivalent ratio of 0.58. In a period of 16-hour length as well as in 9 24-hour periods, no differences were found among the gels.

When heparin release is observed over 3 arbitrarily chosen time periods, the release rates approach linearity (R=0.96 to 0.99 for 0 to 8 hours, R=0.98 for 8 to 96 hours, and R=0.91 to 0.97 for 4 to 10 days). The gel with the lowest calcium:alginate ratio released less heparin in the first 8 hours. Between 8 and 96 hours the release rates were similar for all gels. After 96 hours, the gels with lower amounts of calcium started to release heparin faster than gels with more calcium. By 24 hours, between 35.7 and 46% of the heparin had been released. At 10 days, the gels retained 5.5% to 9.6% of the heparin that was initially immobilized in the gel.

HPLC is an analytical method that uses a high pressure pump to pump a solvent through a column to separate chemical compounds. A detector (ultraviolet, refractive index or post column manipulations) is needed to detect the compounds as they come off the column. The use of the pump allows a high pressure to be built up in the column. This causes a more rapid separation than traditional chromatographic methods allow. By manipulating the solvents in the system, a specific separation of compounds can be achieved. The column we chose was a size exclusion column that allowed us to separate the heparin from the alginate on the basis of their molecular weights. Previous studies of heparin with HPLC have separated the individual molecules in unfractionated heparin using complex combinations of solvents and post-column derivitization. Our separation was simpler because we wanted to separate unfractionated heparin from alginate. The difference between heparin and alginate is that heparin has a molecular weight in the range of 6000 to 30,000 Daltons while alginate has a molecular weight in the range of 240,000 Daltons. In size exclusion chromatography, the molecules with the larger molecular weights come off the column first.

Gel formation characteristics are described in terms of flowability and mobility as summarized in Table 2. There is a large difference in these gel formation characteristics. When less calcium is used to cross-link the alginate, it takes longer for the gels to become solid. Of the gels that formed within 24 hours, the gels with 0.58 calcium to alginate ion equivalence ratio took the longest (10 minutes) to lose their flowability. The gels cross-linked with the 1.2 calcium to alginate ion equivalence ratio took 1 minute to lose their flowability. In theory, rapid gel formation would prevent loss of heparin to the surrounding tissue while the gel was still a liquid. However a slower gelation time allows more time for percutaneous peri-adventitial delivery. When the gel remains liquid for 10 minutes (0.58 gel), the alginate and the calcium are easily injected through a single needle. Furthermore, in its liquid state, the gel is able to track along the vascular sheath to obtain a more uniform distribution along blood vessels.

Immobilization is the ability of the cross-linked alginate to entrap heparin. This entrapment slows the diffusion of heparin from the alginate and protects the heparin from exposure to normal metabolic processes. This is the basis for the sustained release of heparin from alginate. Gels cross-linked with less calcium immobilize more heparin than those cross-linked with more calcium. This is reflected in the amount of heparin recovered from the initial wash of the gel (Table 2).

Gel shrinkage and diffusion are two competing driving forces affecting gel formation, immobilization and heparin release from alginate gels. It has been established that alginate gels shrink during formation. Gels formed with more calcium have a greater cross-linking density. This causes faster gel formation but also increases gel shrinkage. During shrinkage, water is excluded from the gel. Because heparin is in the aqueous phase, increased shrinkage causes more heparin to be excluded from the gel during formation and while the gel is in contact with an aqueous phase. Diffusion is the movement of a heparin within the liquid phase of the gel. Diffusion is limited by the pore size of the gel. Gels formed with more calcium have a greater cross-linking density limiting their pore size. A smaller pore size limits diffusion, retaining more heparin in the gel.

Immobilization and gel formation are dominated by gel shrinkage. During gel formation, shrinkage is maximal. Gels formed with less calcium have lower cross-linking density so shrink less. These are the gels that form slower and retain more heparin within them. Shrinkage is also dominant within the first 8 hours of heparin release. In this time period, the gels with less calcium shrink less, and so exclude less heparin, even though their pore sizes are larger. From 8 to 96 hours, the processes of shrinkage and diffusion are in balance. The gels with less calcium shrink less, and so exclude less heparin, but the gels with more calcium have smaller pore sizes, so they lose less heparin on the basis of diffusion. After 96 hours, diffusion is the predominant process. Because the pore sizes are larger in the gels formed with less calcium, they lose heparin more rapidly after 4 days. Also, by this time, the gels formed with less calcium have more heparin in them, so the gradient between the gel and the liquid is greater for these gels.

Statistical analysis demonstrated that gel with the 0.58 ion equivalence ratio had the slowest release rate over the 10-day observation period. Analysis of variance demonstrated the greatest differences in the initial 8 hours (p=0.0013 for the first 2 hours and p=0.014 for hours 2 through 8). Between 5.5% and 9.6% of the heparin initially present in the gel remains after 10 days. This study demonstrates that alginate gels can release heparin over 10 days.

Our method involved a 24 waiting period prior to the initial wash. That period, chosen for convenience (our study showed that the studied gels actually became solid within an hour of cross-linkage), assured that the gel would be solid prior to evaluation of its kinetics. The kinetics cannot, therefore, be directly transferred to the in vivo model. In transferring this model from the to the animal there will also be a difference in the surface area to which the gel is exposed. In the animal, the gel tracks along the blood vessel, forming a thin layer with a large surface area. In our in vitro experiments, only a small portion (415.3 mm²) of the gel was exposed to the aqueous layer. We do not consider these differences to be major problems, because the purpose of the experiment was only to identify ideal characteristics of the heparin-alginate gel. By choosing the gel with the greatest immobilization and slowest release rate in vitro, the greatest relative immobilization and slowest relative release rate in vivo can be obtained, despite the differences between the in vivo and in vitro systems.

In summary, the gel created with a calcium to alginate ion equivalence ratio of 0.58 had the slowest formation time but it immobilized the most heparin in its matrix. Also, this gel had the slowest release rate over the 10 day observation period and the slowest initial release rate. For these reasons, we used a gel created with 4000 units of heparin (0.2 ml), 1.6 mEq of sodium alginate (1.6 ml, 2% solution), and 0.093 mEq of calcium gluconate (0.2 ml 10% solution) in subsequent animal studies. This gel had a total volume of 2 ml and was easily injected through a 20 gauge needle to the periadventitial surfaces of blood vessels. Accordingly, the percutaneous, peri-adventitial delivery of heparin at the site of angioplasty provides a mechanism for sustained delivery of heparin to reduce intimal hyperplasia.

All of the references cited herein, including patents, patent applications, and publications, are hereby incorporated in their entireties by reference.

While this invention has been described with an emphasis upon certain preferred embodiments, it will be apparent to those of ordinary skill in the art that variations in the preferred compositions and methods may be used and that it is intended that the invention may be practiced otherwise than as specifically described herein. Accordingly, this invention includes all modifications encompassed within the spirit and scope of the invention as defined by the following claims.

What is claimed is:

1. A composition comprising:
a biologically active substance; and
a matrix capable of forming in vivo, said matrix comprising a biologically-compatible polymer having at least two charged portions of the same charge, the polymer cross-linked with at least one biologically-compatible multi-valent counter-ion, wherein said biologically active substance is contained within said matrix.

2. The composition of claim 1, wherein the charged portions of the same charge of the polymer are negatively charged.

3. The composition of claim 2, wherein the polymer is an alginate.

4. The composition of claim 3, wherein the alginate is sodium alginate.

5. The composition of claim 1, wherein the multi-valent counter-ion has a positive charge.

6. The composition of claim 1, wherein the multi-valent counter-ion is divalent.

7. The composition of claim 1, wherein the multi-valent counter-ion is provided by the biologically active substance.

8. The composition of claim 7, wherein the biologically active substance is a protein.

9. The composition of claim 1, wherein the multi-valent counter-ion is provided independent of the biologically active substance.

10. The composition of claim 9, wherein the multi-valent counter-ion is selected from the group consisting of calcium, magnesium, manganese, and combinations thereof.

11. The composition of claim 9, wherein the multi-valent counter-ion is provided by a salt.

12. The composition of claim 11, wherein the salt is a calcium salt.

13. The composition of claim 12, wherein the calcium salt is calcium gluconate.

14. The composition of claim 1, wherein said biologically active substance is selected from the group consisting of a drug, gene, protein, antibody, fatty acid, carbohydrate, vector, cell, nucleic acid, and combinations thereof.

15. The composition of claim 14 wherein the drug is an anti-cancer agent.

16. The composition of claim 15, wherein the anti-cancer agent is selected from the group consisting of paclitaxel, cisplatinum, adriamycin, and combinations thereof.

17. The composition of claim 14, wherein said carbohydrate is a polysaccharide.

18. The composition of claim 17, wherein said polysaccharide is a glycosaminoglycan.

19. The composition of claim 14, wherein said carbohydrate is a starch.

20. The composition of claim 14, wherein said carbohydrate is selected from the group consisting of sucrose, glucose, lactose, maltose, fructose, cellobiose, and combinations thereof.

21. The composition of claim 14, wherein said vector is selected from the group consisting of an adenovirus, plasmid, retrovirus, and combinations thereof.

22. The composition of claim 14, wherein said cell is selected from the group consisting of a natural killer cell, T cell, B cell, red blood cell, white blood cell, macrophage, and combinations thereof.

23. The composition of claim 14, wherein said fatty acid is a triglyceride.

24. The composition of claim 14, wherein said fatty acid is a lipoprotein.

25. The composition of claim 24, wherein said lipoprotein is HDL.

26. The composition of claim 14, wherein said biologically active substance is heparin or a derivative thereof.

27. The composition of claim 14, wherein said biologically active substance is a protein.

28. The composition of claim 27, wherein said biologically active substance is a chemotactic protein.

29. The composition of claim 28, wherein said biologically active substance is a monocyte chemotactic protein.

30. The composition of claim 27, wherein the protein is an angiogenic protein.

31. The composition of claim 30, wherein the angiogenic protein is a vascular endothelial growth factor.

32. The composition of claim 14, wherein said antibody is an anti-vascular endothelial growth factor.

33. The composition of claim 1, wherein said multi-valent counter-ion:polymer IE ratio is from about 0.2 to about 2.

34. The composition of claim 33, wherein the multi-valent counter-ion:polymer IE ratio is from about 0.25 to about 1.2.

35. The composition of claim 34, wherein the multi-valent counter-ion:polymer IE ratio is from about 0.25 to about 0.8.

36. The composition of claim 35, wherein the multi-valent counter-ion:polymer IE ratio is about 0.58.

37. The composition of claim 1. wherein the biologically active substance is a low molecular weight heparin selected from then group consisting of nadroparin or derivatives thereof, enoxiparin or derivatives thereof, and combinations thereof.

38. A composition comprising:
a biologically-active substance; and
a matrix capable of forming in vivo said matrix comprising an alginate cross-linked with at least one biologically-compatible multi-valent counter-ion, wherein said biologically-active substance is contained within said matrix.

39. The composition of claim 38, wherein said alginate is provided by sodium alginate.

40. The composition of claim 38, wherein said multi-valent counter-ion is calcium.

41. The composition of claim 40, wherein said calcium is provided by calcium gluconate.

42. The composition of claim 38, wherein said biologically-active substance is selected from the group consisting of a drug, gene, protein, antibody, fatty acid, carbohydrate, vector, cell, nucleic acid, and combinations thereof.

43. The composition of claim 42, wherein said biologically-active substance is heparin or a derivative thereof.

44. The composition of claim 43, wherein the multi-valent counter-ion:alginate IE ratio is from about 0.20 to about 2.

45. The composition of claim 44, wherein the multi-valent counter-ion:alginate IE ratio is from about 0.25 to about 1.2.

46. The composition of claim 45, wherein the multi-valent counter-ion:alginate IE ratio is from about 0.25 to about 0.8.

47. The composition of claim 46, wherein the multi-valent counter-ion:alginate IE ratio is about 0.58.

48. A method of preparing a composition, said method comprising:
providing a mixture comprising a biologically active substance and a biologically-compatible polymer having at least two charged portions of the same charge; and
cross-linking said polymer with at least one biologically-compatible multi-valent counter-ion to produce a solid matrix capable of forming in vivo, and which contains said biologically active substance.

49. The method of claim 48, wherein the biologically active substance and polymer are combined while each is in liquid form.

50. The method of claim 49, wherein the polymer is dissolved in water.

51. The method of claim 48, wherein the multi-valent counter-ion is combined with the biologically-compatible polymer immediately prior to being percutaneously delivered to an internal locus of a patient.

52. The method of claim 51, wherein the matrix is formed in vivo.

53. The method of claim 48, wherein the multi-valent counter-ion is combined with the biologically-compatible polymer in vivo after being percutaneously delivered to an internal locus of a patient.

54. The method of claim 48, wherein the matrix is formed ex vivo.

55. A method of preparing a composition, said method comprising:
providing a solution comprising heparin and an alginate; and
cross-linking said alginate to produce a solid matrix capable of forming in vivo, and which contains said heparin.

56. The method of claim 55, wherein said alginate is cross-linked with a multi-valent counter-ion.

57. The method of claim 56, wherein the multi-valent counter-ion is calcium.

58. The method of claim 57, wherein said calcium is provided by calcium gluconate.
